(19) United States
(12) Patent Application Publication
(10) Pub. No.: US 2012/0109144 A1
(43) Pub. Date: May 3, 2012

(54) Systems and Methods for Tissue or Organ Removal

(75) Inventors: Albert K. Chin, Palo Alto, CA (US); Lishan Aklog, Scottsdale, AZ (US); Brian deGuzman, Paradise Valley, AZ (US)

(73) Assignee: Pavilion Medical Innovations

(21) Appl. No.: 13/247,688

(22) Filed: Sep. 28, 2011

Related U.S. Application Data
(60) Provisional application No. 61/387,193, filed on Sep. 28, 2010.
(60) Provisional application No. 61/413,587, filed on Nov. 15, 2010.

(51) Int. Cl. A61B 17/26

(52) U.S. Cl. 606/114

(57) ABSTRACT

A system for tissue or organ removal is provided. The system has an outer bag having an exit end, and an inner bag situated within the outer bag and having an opening capable of receiving an organ or tissue. The inner bag may be sealed to the exit end of the outer bag about its opening. A fluid-tight space may be situated between the outer bag and the inner bag. The space may be designed to accommodate positive pressure which can act on the inner bag to cause the inner bag to evert and expel the organ or tissue. The system may also include an organ receiving component for receiving the organ as it is expelled. Methods for tissue or organ removal is also provided.

### RELATED APPLICATIONS

This application claims priority to and benefit of U.S. Provisional Application No. 61/387,192 (filed Sep. 28, 2010) and U.S. Provisional Application No. 61/413,587 (filed Nov. 15, 2010), both of which are incorporated herein by reference in their entirety.

### TECHNICAL FIELD

This invention relates generally to systems and methods for tissue/organ removal, and more particularly, to the use of laparoscopic bags for tissue/organ removal.

### BACKGROUND

Typically, for endoscopic operations (laparoscopy), the abdominal cavity is not opened by a large incision. Rather, instruments can be pushed through the abdominal wall using a trocar, or inserted through a small incision, and actuated from outside the abdominal cavity. To the patient, this surgical method can be less traumatizing than operations involving opening of the abdominal cavity and can result in less discomfort to the patient, less pain, and quicker recovery time. Small pieces of tissue can also be removed directly through the trocar or trocar site (e.g., via a port inserted therein).

In practice, a bag can be inserted through the trocar or a small abdominal incision (e.g., a 20 mm-25 mm incision) into the abdominal cavity to collect the resected tissue/organ prior to removal from the abdomen. This is done to reduce contamination of the abdomen by the tissue/organ (e.g., bowel) during removal. Contamination can be in the form of bacteria, if the tissue/organ is infected, or malignant cells, if the tissue/organ is cancerous. Even with non-infected or benign tissue, it is desirable to insert the tissue into a bag for removal from the abdomen, to avoid misplacement of tissue or tissue segments within the abdominal contents. Misplacement of resected tissue can easily occur, due to the presence of the intestine and other organs and structures within the abdominal cavity.

Laparoscopy bags (“lap-bags”) have been developed for tissue/organ removal. These bags are typically made of a flexible, pliable, and/or durable material, which can be closed by pulling strings at their upper open end. The bags, at present, are designed to hang from the strings and are introduced into the abdominal cavity through the trocar, where they can be opened in the abdominal cavity for placement of the tissue to be removed into the bag. Afterwards, the bag can be closed by pulling the strings and can be withdrawn from the abdominal wall together with the trocar. Thereafter, the tissue can be dissected and the individual lumps can be taken out of the bag, whereupon the bag can be discarded.

This surgical practice can be limited when large size tumors or large masses of tissue/organ are removed from the abdominal cavity. If such material cannot be removed in bulk through the trocar or small abdominal incision, the tumor and the inflamed tissue must be cut or dissected within the abdominal cavity, and can give rise to a contamination of the latter and can present a danger to the patient because of the possibility of metastases. Disruption of tissue inside a lap-bag using hand instruments, such as scissors and forceps, can be tedious and time-consuming, as it is difficult to advance the instruments through the small incision into the intra-abdominal bag to disrupt the tissue. Devices such as morcellators (e.g., a machine with a rotating blade) for cutting tissue into smaller pieces are available for use.

Although laparoscopy bags constitute a considerable improvement, and decrease the chance that impurity can get into the abdominal cavity, the withdrawal of the resected and dissected tissue can be complicated and time-consuming. In addition, danger still exists during withdrawal of the dissected tissue. For example, particles can be introduced into the cavity and, in the case of malign tumors, can give rise to metastases. Furthermore, using a morcellator to dissect the tissue often disrupts the lap-bag, and can cause spillage and contamination inside the abdomen, which may lead to peritonitis (infection of the peritoneal lining of the abdomen), sepsis (infection of the bloodstream) and even death.

Accordingly, it would be desirable to have an effective system for controlled laparoscopic tissue/organ removal that can safely and effectively remove organs of various sizes while minimizing harm to the patient.

### SUMMARY OF THE INVENTION

The present invention provides, in one aspect, an organ or tissue removal system. The system includes, in one embodiment, an outer bag having an exit end. The system can further include an inner bag capable of receiving an organ or tissue, and situated within the outer bag. The inner bag may be sealed, about its opening, to the outer bag at its exit end. The system may also include a substantially fluid-tight space between the inner and outer bag that can accommodate a positive pressure. The pressure, when introduced into the space, can act on the outer bag and cause the outer bag to evert and expel the organ or tissue.

The present invention, in another aspect, features a system for organ or tissue removal. The system can include an apparatus having an outer bag and an inner bag capable of accommodating the organ or tissue. The apparatus, in one embodiment, may include a substantially fluid-tight space between the inner and outer bags. In some embodiments, the system may also include a mechanism for introducing positive pressure into the fluid-tight space, so as to evert the inner bag and expel the organ or tissue from therewithin. The system can also include a receiving apparatus for receiving the organ or tissue as it is expelled from the inner bag. In some embodiments, the system can include a cutting mechanism that can be used to cut the organ or tissue into relatively smaller pieces as it is expelled from the inner bag.

In another embodiment, the present invention provides a method for removing an organ and/or tissue from a body. The method may include positioning, adjacent to a site of an incision, an apparatus having an outer bag, an inner bag situated within the outer bag, and a substantially fluid-tight space between the outer and inner bag. Organ or tissue to be removed may thereafter be placed within the inner bag. Subsequently, a positive pressure may be applied within the fluid-tight space between the outer and inner bags so that the inner bag can evert and expel the organ or tissue through the site of incision.

The method may also include positioning an organ receiving apparatus opposite the inner bag, in order to receive the organ as it is expelled from the inner bag. In some embodiments, the organ or tissue may also be cut into relatively smaller pieces during eversion of the inner bag.

### BRIEF DESCRIPTION OF DRAWINGS

FIGS. 1a-1b illustrates an organ removal system in accordance with an embodiment of the present invention.

FIGS. 2a-3c illustrate an organ removal system in accordance with a second embodiment of the present invention.

FIGS. 3a-3c illustrate use of the organ removal system of FIGS. 2a-2c in accordance with an embodiment of the present invention.

FIG. 4 illustrates an organ removal system in accordance with a third embodiment of the present invention.

FIGS. 5a-5c illustrate use of the organ removal system of FIGS. 1a-1c in accordance with an embodiment of the present invention.

### DESCRIPTION OF SPECIFIC EMBODIMENTS

In accordance with one embodiment of the present invention, a system is provided herein for tissue and/or organ removal. In some embodiments, the invention may be used in connection with laparoscopic or minimally invasive surgery. The system, as described below, can be used to remove various tissues and/or organs from a body. As used herein, the terms “organ” and “tissue” may refer to bodily organs, portions of bodily organs, body tissue, or any other type of bodily tissue, bodily fluid, or body material.

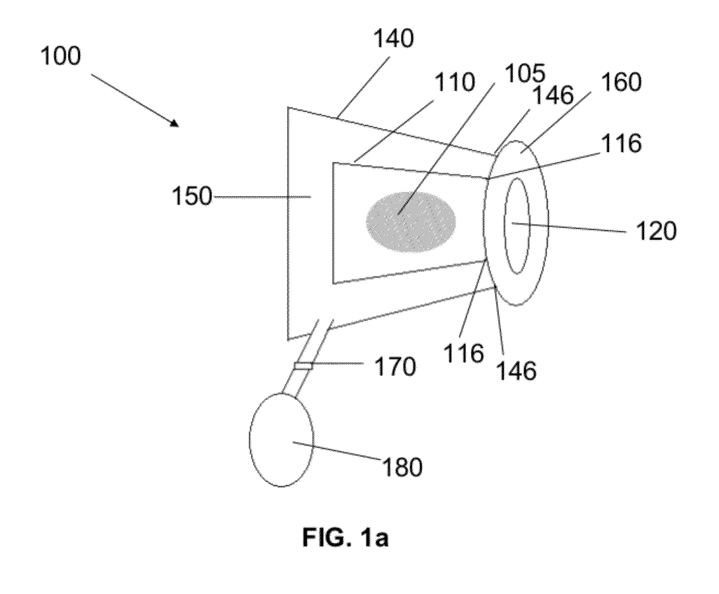

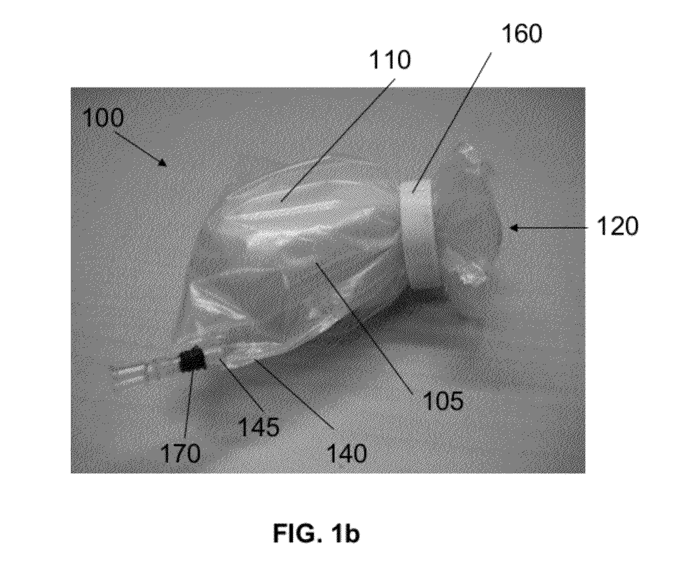

FIGS. 1a-1b show an apparatus 100 for organ and/or tissue removal according to one embodiment of the present invention. As illustrated in FIG. 1a, apparatus 100, in an embodiment, can include an inner bag 110, an outer bag 140, and a substantially fluid-tight space 150 between the outer bag 140 and the inner bag 110.

The inner bag 110, in an embodiment, can be designed to accommodate an organ and/or tissue 105 therewithin, and to deliver the organ 105 from a first location to a second location. In one instance, the inner bag 110 can delivery the organ 105 from a first location at a point within the body of a patient to a second location at a point outside the body of the patient. In some instances, the inner bag 110 can deliver the organ to the point outside the body through a laparoscopic incision or puncture in the skin.

As illustrated, the inner bag 110 can include an opening 120 through which the organ 105 within a patient can be placed into the inner bag 110 and through which the organ 105 can subsequently be expelled from the body of the patient. In one embodiment, the opening 120 can be designed for placement adjacent to a site of incision. By being placed at or near the site of incision, the inner bag 110 can conveniently expel the organ 105 through the opening 120, as well as through the site of incision and out the body.

To move the organ 105 through the site of incision, the inner bag 110 can be moved from a resting state where it accommodates a bodily organ 105 therewithin, to an everted state, where it is substantially pushed through opening 120, in response to a force (e.g. a positive pressure) being applied to the inner bag 110 to displace the bodily organ 105 through the opening 120.

In an embodiment, with the organ 105 situated within the inner bag 110, the inner bag 110 can be compressed in such a manner that the inner bag 110 forms a substantially tight hold on the organ 105 and conforms to substantially the profile of the organ 105. In this compressed state, the inner bag 110 can provide a substantially strong grasp on the organ 105 in such a manner so as to subsequently allow the organ 105 to be smoothly expelled from within the inner bag 110 and through the incision. In certain instances, the compression of the inner bag 110 can act to partially compress or otherwise reduce the size or volume of the organ 105 to permit the organ to better fit through the opening 120 and the site of incision.

To aid in removal of the organ 105 from the body, the inner bag 110 can be made from a material that is flexible and/or pliable and capable of withstanding a sufficient force without rupturing. The material of the inner bag 110, in another embodiment, can be impermeable to fluids in order to allow a fluid to exert a force on the inner bag 110. Since the inner bag 110 is designed to be inserted into the body of a human or animal, the inner bag 110 can also be made from a material that is biocompatible. The biocompatibility of the material can help minimize occurrence of adverse reactions due to use of the inner bag 110 within the body.

The inner bag 110 can further be made from any material that can aid in the passage of the organ 105 out of the inner bag 110. In one embodiment, the inner bag 110 can be made from a material that minimizes resistance and friction between the inner bag 110 and the organ 105, so as to allow the organ 105 to be easily removed. In another embodiment, the inner bag 110 can include a coating that minimizes resistance and friction. For instance, the inner bag 110 can be made from a material or have a coating that is substantially smooth and/or has a relatively low coefficient of friction so that organ 105 can more easily pass through opening 120.

The size of the inner bag 110, in an embodiment, can vary depending on a variety of characteristics. In certain instances, the size of the inner bag 110 can be dependent on the size of the organ being removed. In other instances, the size of the inner bag 110 can be selected based on the location of the organ being removed or the cavity within which inner bag 110 is employed. It should be noted that the size of inner bag 140 may also vary so that it may fit within outer bag 140. To the extent desired, the inner bag 110 can be smaller in size, the same size, or larger in size than the outer bag. In some cases, if inner bag 110 is larger than outer bag 140, inner bag may be flexible, pliable, and/or foldable to allow it to be situated within outer bag 140,

The inner bag 110 can have any shape desirable so long as the shape allows the inner bag 110 to fit within the outer bag 140. In one embodiment, the inner bag 110 can have a bag shape. Of course, other geometric shapes can also be possible, including, but not limited to, a cone, a cylinder, a rectangle, etc. In some embodiments, inner bag 110 may have a shape designed to aid removal of a particular organ having a particular shape.

The organ removal apparatus 100 can further include, in accordance with an embodiment of the present invention, the outer bag 140. As shown in FIG. 1a, the outer bag 140 can be situated about the inner bag 110. In one embodiment, the outer bag 140 can be made from any sufficiently flexible or pliable material that allows the outer bag 140 to be accommodated within a body. For instance, when the outer bag 140 enters the body, the outer bag 140 can be floppy and/or foldable to allow the outer bag 140 to navigate to a site of interest. The outer bag 140 may also be constructed from a material having sufficient strength to withstand fluid pressure introduced into the bag without rupturing. The outer bag 140, in an embodiment, should further be made from a material that is substantially inelastic and/or non-expandable so that it can maintain its shape in the presence of fluid pressure within outer bag 140 during the organ removal process. By substantially maintaining its shape while it is filled with fluid, the outer bag 140 can allow for the fluid pressure to be directed toward the inner bag 110, essentially allowing the pressure to “push” inner bag 110, so as to cause eversion of the inner bag 110, and the organ 105.

The size of the outer bag 140, in an embodiment, can vary depending on a variety of characteristics and as long as the size allows the inner bag 110 to fit within the outer bag 140.

The outer bag 140 can have any shape desirable so long as the shape allows the inner bag 110 to fit within the outer bag 140. In one embodiment, the outer bag 140 can have a bag shape. Of course, other geometric shapes can also be possible including, but not limited to, a cone, a cylinder, a rectangle, etc. In some embodiments, inner bag 110 may have a shape designed to aid removal of a particular organ having a particular shape.

Still looking at FIG. 1a, the outer bag 140 and the inner bag 110 can be arranged to define a substantially fluid-tight space 150. The space 150, in an embodiment, can be defined by a seal provided between the opening 120 of inner bag 110 and the exit end 146 of outer bag 140. The space 150 can be designed to allow fluid (i.e., liquid, air, gas, or other substances) to enter so as to increase pressure within the space 150. It should be noted that a substantially incompressible fluid (e.g. a liquid such as water or saline) can be used to facilitate a substantially smooth eversion process because the user can better control the amount of pressure exerted onto inner bag 110. In particular, as pressure is exerted within the space 150, the pressure can act to compress the inner bag 110 onto the organ 105, and subsequently cause the inner bag 110 to evert (i.e., turn inside-out). In other words, when fluid enters the space 150, the fluid is capable of applying a pressure or force to the inner bag 110 to cause the inner bag 110 to move to a compressed state. As fluid continues to enter the space 150, the pressure may increase so as to push the body of the inner bag 110 out through the opening to place it into an everted state. Eversion of the inner bag 110 can occur because the outer bag 140 is sufficiently strong to hold fluid within and to allow fluid to be directed onto the inner bag 110.

The bags 110 and 140, in an embodiment, can be made from substantially the same material or different materials. In some embodiments, any sufficiently flexible or pliable material including various types of plastics (e.g., nylon, polycarbonate, Ultem), memory materials, cloth, silicon, foil, and polymers can be used. The material can be clear or transparent, so that the contents in the bags can be seen from the outside. The material can also be biocompatible to minimize occurrence of adverse reactions. The material can be antistatic, pyrogen-free, non-toxic and/or sterilizable. The material can be substantially impermeable to fluids to minimize spillage and/or contamination. In one embodiment, the bags can be coated or laminated to form a barrier to avoid infected or cancerous cells that might be contained in the excised tissue from coming into contact with the trocar or incision site wound. The bags can also be made from a material, or coated, to reduce friction between the bags and the organ to be removed. The bags can have sufficient durability to withstand manipulation, puncture, and/or rupture. For example, the bags 110 and 140 can be made of a material with sufficient strength enabling the bags to be opened in the abdominal cavity and to retain the opened position.

The apparatus 100 of the present invention can further be provided with an inflation port 170 through which fluids can enter with sufficient pressure to evert the inner bag 110 and remove the organ 105 from within the body. The inflation port 170 can be situated anywhere on the outer bag 140, so long as the inflation port 170 can be accessible to a user of the apparatus 100. In one embodiment, inflation port 170 can be situated at end 145 of outer bag 140. Of course, other locations for the inflation port 170 are possible as long as the location is accessible to the user and as long as fluids can enter with a sufficient force to evert the inner bag 110. Additionally, if desired, inflation port 170 can be provided with a substantially long tube that can extend through the incision to allow easier delivery of fluid through the port 170 and into space 150.

To direct fluid through the inflation port 170 and into the space 150, the organ removal apparatus 100 of the present invention can include a mechanism for infusing fluid into and/or withdrawing fluid from the space 150, such as inflation mechanism 180. As shown in FIG. 1a, the inflation mechanism 180 can be coupled to the inflation port 170 and can be designed to direct fluid into the space 150, inflate outer bag 140, and apply a positive force (i.e. positive fluid pressure) to the inner bag 110. Following inflation, the inflation mechanism 180 can be designed to withdraw fluid from the space 150 so that outer bag 140 can be deflated for removal from the body.

In an embodiment, the inflation mechanism 180 can include, for instance, an inflation catheter or any other inflation device capable of infusing and/or withdrawing fluid from the space 150. In one embodiment, the inflation mechanism can be a pump, a squeeze bag, a hydraulic apparatus, or any other mechanism for infusing fluid into and/or withdrawing fluid from the space 150. The inflation mechanism 180, it should be noted, can be coupled to the inflation port 170 at any location on the outer bag 140 so long as the inflation mechanism 180 can extend through the incision to be operable by the user.

In one embodiment, the inflation mechanism 180 can be detachably coupled to the inflation port 170 on the outer bag 140. As such, the inflation mechanism 180 can be disconnected and detached from the inflation port 170 following inflation or deflation.

Connection of the inflation mechanism 180 to the inflation port 170 can occur, in certain embodiments, through the use of a connector (not shown). The connector can act to couple the inflation mechanism 180 to the inflation port 170, allowing the inflation mechanism 180 to inflate the outer bag 140. In an embodiment, the connector can be situated on either the inflation mechanism 180 or the inflation port 170. Alternatively, the connector can include a two-piece design having two complimentary pieces to permit coupling between the inflation mechanism 180 and the inflation port 170.

The organ removal apparatus 100 can further include, in accordance with an embodiment of the present invention, a framework 160 that can be used to secure inner bag 150 to the site of incision. An example of a framework 160 can be a space occupying inflatable ring, or any other framework 160 with sufficient structural integrity to adequately secure the opening 120 of the inner bag 110 to the site of incision.

As shown in FIG. 1a, the framework 160 can be sealed to the inner bag 110 at its proximal end 116 (e.g. at the opening 120 of the inner bag 110) and to the outer bag 140 at its exit end 146. The seal between framework 160 and proximal tips 116 and 140 may be fluid-tight, so as to define the substantially fluid-tight space 150 between the between the inner bag 110 and the outer bag 140.

The framework 160, in an embodiment, can be designed to provide a structure or frame for maintaining an opening at the site of incision in order to facilitate removal of organ 105 through the incision. Accordingly, the framework 160 can be provided with sufficient structural integrity to maintain the opening at the site of the incision. For example, if framework 160 is a ring (or other type of open frame), framework 160, once placed adjacent to the incision, may be able to hold the incision open during eversion. In another embodiment, framework 160 may be able to hold inner bag 110 in an open position within the patient's body. In this case, the organ 105 may pass through the framework and outside of the body during eversion of the inner bag 110.

If desired, the framework 160 can also be designed to anchor or secure the apparatus 100 at the incision site. Barbs, clips or other attachment mechanisms can be used to aid in anchoring the framework 160 at the site of surgery, for example.

The framework 160, in an embodiment, can be made from a material that is inflatable, flexible, malleable, pliable, and/or expandable. In one embodiment, an anti-inflammatory agent such as dexamethasone, prednisolone, corticosterone, budesonide, estrogen, sulfasalazine, mesalamine, or any suitable combination or mixture thereof can be applied to the framework 160 to prevent inflammation or any other adverse reaction caused by the engagement of the framework 160 adjacent the site of incision.

The framework 160, in an embodiment, can have any size desirable, depending on the particular application, as the size of the framework 160 can depend on the size of the incision or the size of the organ being removed. For instance, a larger framework 160 can be necessary for placement adjacent to a larger incision, and/or to allow a larger organ 105 to pass through. It should be noted that the size of the framework 140 can be able to permit the framework 160 to be securely positioned adjacent the site of incision.

The framework 160, in an embodiment, can be toroidal (i.e. doughnut, torus, or other toroid) in shape. A toroid shape can allow the framework 160 to be positioned adjacent the site of incision and can substantially conform to the shape of the organ 105 as the organ 105 is being removed from the body. Of course, other shapes for the framework 160 can be possible.

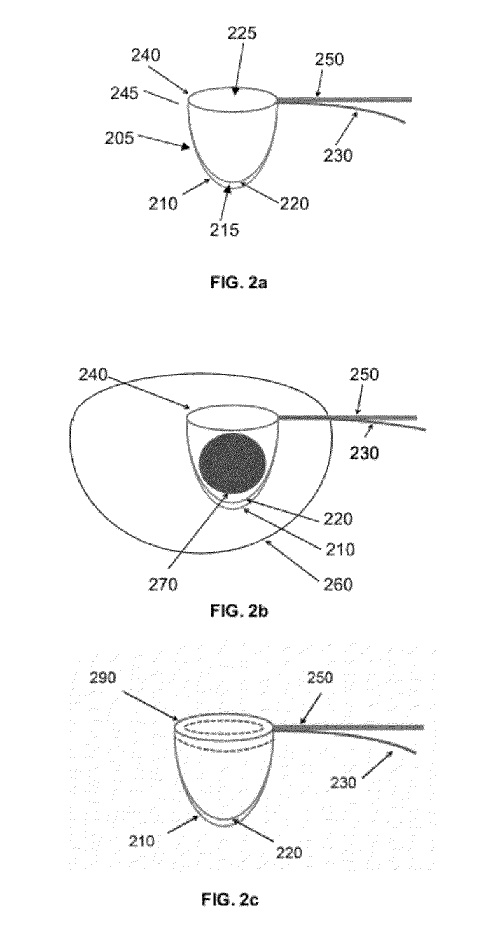

Looking now at FIGS. 2a-2c, there is illustrated a system 200 for tissue/organ removal in accordance with another embodiment of the present invention. Similar to the embodiments described above, the system 200 can include a organ accommodating apparatus, such as a double-walled bag or double bag 205, having an outer bag 210 and an inner bag 220. The inner bag 220, in an embodiment, can have an opening 225 through which a tissue/organ 270 can be accommodated. The bags 210 and 220 can be attached (e.g., molded, sutured, pressed, closed, sealed, etc.) near the opening 225 of inner bag 220 and the exit end 245 of outer bag 210 to form a fluid-tight seal between the outer bag 210 and inner bag 220. In one embodiment, the double bag 205 can be molded as a one-piece unit. In some examples, the double bag 205 can be formed by folding a portion of a balloon-shaped member (e.g., round or elliptical) inwardly, e.g., by pushing one end of the member toward an opposing end.

There also can be a space 215 situated between the outer bag 210 and the inner bag 220. The space 215 can be substantially fluid-tight to minimize fluid spillage upon introduction of a fluid into the space 215. Suitable fluids include, for example, gasses (e.g., air) and liquids (e.g., water, saline). The space 215 may be designed so that, upon introduction of a fluid into the space 215, the fluid can exert a sufficient force onto the inner bag 220 to evert the inner bag 220 and displace the tissue/organ 270 from within the inner bag. In some embodiments, substantially incompressible fluid (e.g., water, saline, etc.) is used for inflation so that a user can control the amount of pressure exerted on inner bag 220. In certain embodiments, compressible air or gas inflation can also be used.

In an embodiment, an inflation line 230 can be attached to the double bag 205 to provide fluid communication with the space 215. The inflation line 230 can be a port or conduit for directing a fluid flow into and out of the space 215. The inflation line 230 can be attached to the double bag 205 near its opening 225 or at other suitable position as long as the distal end of the inflation line is accessible to the user. The line 230 can extend between the outer bag 210 and the inner bag 220 to ensure that the inflation path is not substantially compromised at the entrance site of the bag 205 into the abdomen. If desired, inflation line 230 can be provided with a substantially long tube that can extend through the incision to allow easier delivery of fluid through the line 230 and into the space 215.

As illustrated in FIGS. 2a-2b, a framework, such as an elastic, expandable, and/or inflatable ring 240, can be placed adjacent to the opening 225 to facilitate the attachment of the bags 210 and 220, the sealing of the space 215, and/or the connection with the inflation line 230. The elastic ring 240, in an embodiment, can have a toroid shape or any other shape that can allow the contents (e.g., tissue/organ 270) in the inner bag 220 to pass through. The elastic ring 240 can be sufficiently expandable and/or inflatable, such that the elastic ring 240 can substantially conform to the shape of the tissue/organ being removed from the body, and/or that tissues/organs of different sizes can pass through. In an embodiment, the elastic ring 240 can be made from a material that is sufficiently flexible or pliable so that it can be folded, expanded or otherwise manipulated when introduced into the body, and also sufficiently rigid to provide adequate support during use.

The elastic ring 240, in one embodiment, can be a separate component attached to the opening 225 of inner bag 220. For example, elastic ring 240 may be attached by loops (e.g. loops similar to belt loops), or a sleeve. This may allow elastic ring 240 to be replaced and/or removed if desired. In another embodiment, elastic ring can be integral to and/or molded to the opening 225 of inner bag 220 and/or outer bag 210. Of course, elastic ring 240 may also be attached to inner bag 220 and/or outer bag 210 in other ways.

As discussed above, the elastic ring 240 can also be used to secure the opening 225 of the inner bag 220 adjacent to the site of incision. The elastic ring 240, in an embodiment, should have sufficient structural integrity for adequate support and securing near the site of incision. Barbs, clips or other attachment mechanisms can be used to aid in securing the elastic ring 240 at the site of incision. In one embodiment, an anti-inflammatory agent such as dexamethasone, prednisolone, corticosterone, budesonide, estrogen, sulfasalazine, mesalamine, or any suitable combination or mixture thereof can be applied to the elastic ring 240 to prevent inflammation or any other adverse reaction caused by the engagement of the elastic ring 240 adjacent the site of incision.

The elastic ring 240, in an embodiment, can have any size desirable, depending on the particular application, the size and weight of the double bag 205, the size of the incision, and the size of the tissue/organ being removed. For instance, a larger elastic ring 240 can be used to allow a larger organ to pass through.

Referring now to FIG. 2b, the double bag 205 and elastic ring 240 can be introduced into the abdominal cavity 260 through a small incision or trocar port (not shown). In some embodiments, the double bag 205 may be introduced through a lumen of a trocar. However, if an incision is large enough, the double bag 205 may be introduced into the abdominal cavity through the incision without the use of a trocar.

A handle 250 can be attached to the double bag 205 to assist insertion and/or withdrawal of the double bag 205 and elastic ring 240. The handle 250 can be attached to the double bag 205 at substantially the same position as the inflation line 230. For example, the handle 250 and inflation line 230 can be both attached to the elastic ring 240 at the same position or adjacent to each other. In one embodiment, the handle 250 can include at least a portion of the inflation line 230 therein, or the inflation line 230 can be incorporated in the handle 250. For example, the handle 250 can include a cannula that allows a fluid flow therein. In some embodiments, the handle 250 and inflation line 230 can be attached to the double bag 205 at different positions.

In an embodiment in accordance with the present invention, the double bag 205 can be packed inside a sheath for delivery before being introduced into the abdominal cavity 260 to keep the double bag 205 in a substantially compressed or collapsed state, so as to facilitate insertion. The sheath, if desired, can be withdrawn after insertion of the double bag 205 into the abdomen cavity 260. In one embodiment, the elastic ring 240 can also be packed in the sheath in a substantially compressed or folded state before the double bag 205 is inserted into the abdomen. Once inserted inside the abdomen, the sheath can be removed and the elastic ring 240 can expand to an open position to facilitate accommodation or placement of the tissue or organ 270 into the double bag 205.

As shown in FIG. 2c, the system 200 can further include an inflatable ring 290. In one embodiment, the elastic ring 240 can be substituted by the inflatable ring 290. In another embodiment, the elastic ring 240 can be used in conjunction with the inflatable ring 290. The inflatable ring 290 can be inflated upon introduction of fluid via the inflation line 230 (or via another line, not shown), so as to open the opening 225 of the inner bag 220 for tissue or organ insertion. Once inflated, the ring 290 can have sufficient structural integrity to adequately hold the opening 225 open. The ring 290 can be made from a material that is inflatable or expandable (e.g., plastics, memory materials, silicon, etc.).

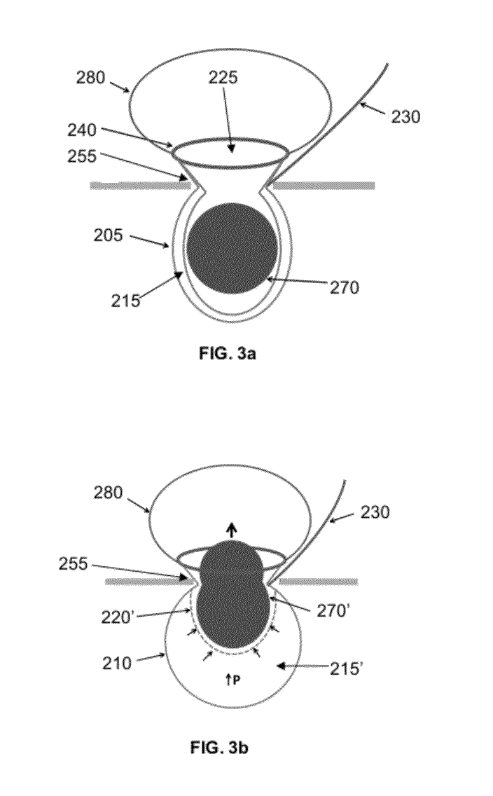

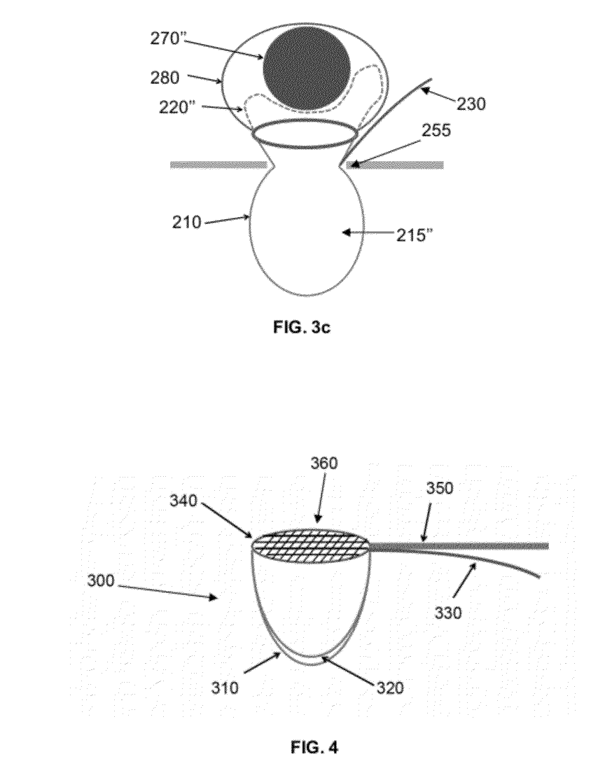

Turning now to FIG. 3a, once the tissue or organ 270 has been placed inside the double bag 205, the opening 225, by design, can be withdrawn from the abdominal cavity 260, e.g., through the incision 255. To this end, should it be desired, an organ receiving apparatus (for example, bag 280) may be placed external to the body for receiving the tissue/organ 270 being removed from the body. The use of bag 280 can prevent or minimize spillage of infected, malignant, or otherwise undesirable tissue onto the surface of the body.

In some instances, bag 280 may be placed about the opening 225 to receive the organ or tissue as it is expelled. In one embodiment, to allow advancement of the opening 225 through the incision 255, the elastic ring 240 can be compressed or folded. The elastic ring 240 can then reopen and be stabilized when the opening 225 is outside the incision 255. In embodiments where the inflatable ring 290 is used, the inflatable ring 290 can be deflated to allow the opening 225 to be advanced from the abdomen, then be re-inflated outside the abdomen to provide structure and support as needed.

It should be noted that in some embodiments, the opening 225 needs not be advanced from the abdominal cavity 260 for expelling the tissue/organ 270 from the inner bag 220. Rather, the opening 225 can remain substantially inside the abdominal cavity 260, and bag 280 can be inserted into the abdominal cavity 260 and placed over opening 225 to receive the tissue/organ 270. Alternatively, the opening 225 can be positioned adjacent to the abdominal incision 255 (e.g., at substantially the same level as the skin/wound edge) so that an external bag can be used to receive the tissue/organ 270.

The external bag 280, when in use, can be placed in substantially opposing relation to the inner bag 220. In one embodiment, the exit end 245 of the external bag 280 can be attached to the elastic ring 240 and/or inflatable ring 290, over the opening 225 of the inner bag 220. The attachment between the external bag 280 and the double bag 205 can be substantially fluid tight, so as to minimize spillage of any infected or malignant contents. The external bag 280 can be made from the same or different material than the outer and inner bags 210 and 220.

Once at the site of interest, the apparatus 100 can be prepared for removing organ 105. Applying a force onto the inner bag 110 can act to deform the inner bag 110 in such a manner as to evert the inner bag 110 and expel the organ 105 from within, as shown in FIGS. 3b and 3c. Following eversion of the inner bag 110 and removal of the organ 105, the system 110 can be removed from the body.

In accordance with an embodiment of the present invention, the above described apparatuses, systems, and designs can further include a cutting member adapted to cut the tissue/organ into pieces. As illustrated in FIG. 4, the system 300 can include a grate 360 to cut the tissue/organ as the tissue/organ is expelled from the inner bag 320. For example, as the positive pressure everts inner bag 110, the organ or tissue may be pushed through grate 360 and cut into smaller pieces. The grate 360 can be positioned near the elastic ring 340 or inside the inner bag 320 after the tissue/organ has been placed inside the inner bag 320. The grate 360 can also be attached to the third bag for receiving the expelled tissue/organ. To the extend desired, grate 360 may be provided with at least one blade to enhance cutting of the tissue. Of course, other cutting mechanism may also be used such as a grater, a series of blades, an apple cutter, etc. In some embodiments, the cutting mechanism may be a powered mechanism, such as a spinning blade, a saw, a scissor, a morcellator, etc.

In operation, an incision may be made at the site of interest. The apparatus 100 can then be inserted into the body through the incision, and advanced through the body to a site of interest for organ removal. In some instances, the apparatus 100 can be inserted into the body through a trocar that has been pushed through the incision. In other instances, the apparatus 100 may be inserted through the incision without the use of a trocar. Other surgical instruments may also be inserted through the trocar or incision for use during the surgical procedure.

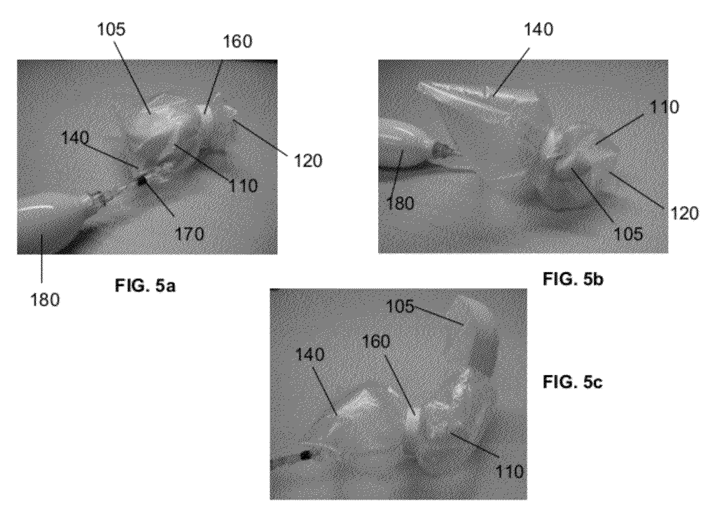

Removal of an organ or tissue can first require inserting the desired organ/tissue 105 into the inner bag 110 of apparatus 100, as shown in FIGS. 3a and 5a. For example, a surgeon may use instruments to cut or resect the organ and place it within inner bag 110. In some embodiments, an elastic ring positioned at the opening 120 of inner bag 110 may hold inner bag 110 in an open position so that the organ 105 can be placed within inner bag 110. Once inside the inner bag 110, the framework 160, in one embodiment, can be inflated and secured to the site of incision (e.g. as shown in FIGS. 3b and 5b). If a trocar is used, the trocar may then be removed from the incision, prior to securing the apparatus 100 to the site of incision, so the organ can be expelled. In one embodiment, the opening 120 (opening 225 as labeled in FIG. 3a) of inner bag 110 may be drawn through the incision so that the opening 120 is positioned outside the body, as shown in FIGS. 3a-3c. Subsequently, the inflation mechanism 180 can be activated, causing fluid to enter the space 150 between the outer bag 140 and inner bag 110 so as to apply a positive pressure onto the inner bag 110. As illustrated in FIGS. 3b-3c, upon introduction of a fluid (e.g., water or saline via the inflation line 230), the space 215 can be inflated and grow in size (e.g., 215′ and 215″). The continual introduction of fluid into the space 215 can exert a positive pressure (indicated by arrows P) onto the inner bag 220′ and displace the tissue/organ 270′ through the incision. When the outer bag 210 is substantially full of the fluid and the space has grown to 215″, both the inner bag 220″ (in a substantially everted position) and the tissue/organ 270″ (in a substantially expelled position) are delivered out of the incision and into the external bag 280.

In some embodiments, an organ receiving apparatus, as shown in FIG. 3a-3c, 280 can be placed around the opening 225 of inner bag 220 and/or outer bag 210 to receive organ 270 as it is expelled. After delivery of the tissue/organ 270″ into the organ receiving apparatus 280, the organ receiving apparatus 280 can be detached from the inner bag 220 and/or outer bag 210, which can then be deflated and withdrawn from the abdominal cavity. Alternatively, the external bag 280 can remain attached to the double bag 205 while the double bag 205 is being deflated and withdrawn from the abdominal cavity.

Although described as using one incision, multiple incisions may also be used if it is so desired. For example, the apparatus 100 may be introduced into the body through one incision, and then used to expel the organ through another incision. Additional incisions may also be used as required by the surgery.

Also, one skilled in the art will recognize that, although a particular sequence of using apparatus 100 is described, the sequence is not intended to be limiting. In different embodiments, the order of the various steps described above may be altered to facilitate removal of the organ or tissue. Additionally, some steps may be added or removed as desired.

It should be appreciated that while described herein as removing tissues/organs, the system of the present invention can also be used to introduce tissues/organs into the body, for instance, during transplantation. The present invention may also be used to remove foreign bodies from or introduce foreign bodies into the body. It should also be appreciated that the system of the present invention can be used in other disciplines besides medicine. For instance, the system can be used to deliver goods through a portal and/or into hard-to-reach areas.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A system for organ or tissue removal, comprising:
an outer bag having an exit end;
an inner bag situated within the outer bag and having an opening capable of receiving an organ or tissue, the inner bag being sealed about its opening to the exit end of the outer bag;
a fluid-tight space situated between the outer bag and the inner bag, the space designed to accommodate positive pressure which can act on the inner bag to cause the inner bag to evert and expel the organ or tissue.

2. A system as set forth in claim 1, further comprising a framework situated adjacent to the opening for maintaining the inner bag in an open position.

3. A system as set forth in claim 2, wherein the framework includes an inflatable ring.

4. A system as set forth in claim 2, wherein the framework includes an anchor to secure the inner bag at a surgical site.

5. A system as set forth in claim 1, further comprising a port, situated on the outer bag, through which fluid can enter into or be removed from the space.

6. A system as set forth in claim 5, further comprising a tube that can extend from the inflation port to direct fluid into and out of the space.

7. A system as set forth in claim 1, further comprising a mechanism for introducing and removing positive pressure from the space by infusing fluid into and/or withdrawing fluid from the space.

8. A system as set forth in claim 7, wherein the inflation mechanism includes a connector capable of detachably coupling to a port on the outer bag.

9. A system for organ or tissue removal, comprising:
an organ accommodating apparatus having a outer bag and an inner bag for accommodating an organ therewithin, the inner bag being sealed about its opening to an exit end of the outer bag;
a substantially fluid-tight space situated between the first and second bag; and
an organ receiving apparatus for receiving the organ expelled by the inner bag upon eversion.

10. A system as set forth in claim 9, wherein the organ receiving component is a bag designed to be placed in opposing relation to the inner bag and capable of receiving the organ expelled by the inner bag.

11. A system as set forth in claim 9, further comprising a framework situated adjacent to the opening for maintaining the inner bag in an open position.

12. A system as set forth in claim 11, wherein the framework includes an elastic ring.

13. A system as set forth in claim 11, wherein the framework includes an inflatable ring.

14. A system as set forth in claim 11, wherein the framework includes an anchor to secure the inner bag at a surgical site.

15. A system as set forth in claim 9, further comprising a mechanism for providing positive pressure within the space that can cause the inner bag to evert and expel the organ.

16. A system as set forth in claim 15 wherein the mechanism provides the positive pressure by infusing fluid into and/or withdrawing fluid from the space.

17. A system as set forth in claim 15, wherein the inflation mechanism includes a connector capable of detachably coupling to a port on the outer bag.

18. The system of claim 9, further comprising a cutting member situated adjacent to the opening of the inner bag and adapted to cut the organ as it is expelled from the inner bag.

19. A method for organ or tissue removal comprising:
positioning, adjacent to a site of incision, an apparatus for accommodating an organ or tissue to be removed;
placing the organ or tissue to be removed into the inner bag; and
everting the apparatus so as to expel the organ or tissue through the site of incision.

20. A method as set forth in claim 19, wherein, in the step of positioning, the apparatus includes an outer bag, an inner bag situated within and attached to the outer bag, and a substantially fluid-tight space between the outer and inner bag.

21. A method as set forth in claim 20, wherein the step of everting includes introducing a positive pressure within the fluid-tight space in order to evert the inner bag.

22. A method as set forth in claim 19, further comprising situating an organ receiving mechanism opposite the apparatus, and receiving the organ or tissue in the organ receiving mechanism as it is expelled from the apparatus.

23. A method as set forth in claim 19, further comprising cutting the organ or tissue into relatively smaller pieces as it is expelled from the apparatus.

24. A method as set forth in claim 23, wherein the step of cutting includes forcing the organ or tissue through a cutting mechanism as the organ or tissue is expelled from the apparatus.
